(19) United States
(12) Patent Application Publication
(10) Pub. No.: US 2016/0334270 A1
(43) Pub. Date: Nov. 17, 2016

(54) TRANSMISSION WITHOUT REVERBERATION BY ITERATIVE INCOMPLETE TIME-REVERSAL

(71) Applicant: WESTERNGECO LLC, Houston, TX (US)

(72) Inventors: Dirk-Jan Van Manen, Zurich (CH); Ivan Pires de Vasconcelos, Cambridge (GB)

(21) Appl. No.: 15/111,739

(22) PCT Filed: Jan. 13, 2015
(86) PCT No.: PCT/US2015/011194

§ 371 Date: Jul. 14, 2016

Related U.S. Application Data
(60) Provisional application No. 61/927,317, filed on Jan. 14, 2014.

(51) Int. Cl. G01H 3/00; A61B 8/08; G01V 1/36

(52) U.S. Cl. CPC G01H 3/00; G01V 1/364; A61B 8/0866; A61B 8/5207; G01V 2210/679

(57) ABSTRACT

For wavefield analysis/processes, constructing f1 solutions and utilizing the f1 solutions of a medium using iterative incomplete time-reversal. Using sources convolved with the f1 solution, propagating a wavefield without internal reverberation in a medium. Many applications of the resulting reverberation-free wavefield or virtual data are also disclosed.

### BACKGROUND

This disclosure relates to transmission of waves through a medium without reverberation for various industries and, in particular, relates to methods and apparatuses using the f1 solution constructed by iterative incomplete time-reversal.

Wave propagation is present in many different physical systems that are important in many different industries. The study of wave propagation affected by an object in a medium can provide insight of the physical properties or structures of the object. A propagating wave may be used as an information carrier to transmit information from one location to another location.

In a propagating wave system, a source may generate a wave, which propagates through a media and is disturbed by an object. A receiver may measure the disturbed wavefield by capturing some energy from the wave. From the measurements at the receiver and the source, one may derive certain information about the properties of the object, or obtain an image of the object. The wave may be any kind of physical wave, such as electro-magnetic wave (e.g., radio wave or X-ray) or mechanical wave (e.g., ultrasound, acoustic or elastic). The measurements at a certain receiver may contain many limitations and other undesirable or unusable signals (noises). It is desirable to remove noises and avoid limitations of any particular receivers. The measurements at receivers may be processed before they are used to produce images of the object or for other purposes.

Waves propagating in layered media, such as the subsurface in exploration seismic surveys, are normally subject to partial reflection and transmission at the interfaces, where the propagation related medium properties change. In such cases, a single pulse emitted on one side of the medium (e.g., the top in surface seismic applications) can give rise to a train of transmitted reverberations on the other side (e.g., deeper in the subsurface) due to internal multiple reflections. The same internal reverberations also affect the reflected wavefield, which then can have a similar train of arrivals.

Internal reverberations are such a fundamental consequence/expression of wave-propagation in layered media that the effect may be said to constitute normal forward scattering. Nevertheless, internal multiples in the reflected wavefield resulting from/caused by the reverberations complicate processing and interpretation of the wavefield (e.g., for potential hydrocarbon bearing formations) and routine seismic processing still aims to remove such internal multiples from the data before producing an image of the subsurface. It would be desirable to find a way to acquire data without internal reverberations as opposed to remove them in processing and/or imaging.

In other fields, similar situations exist. For example, in the case of communication applications, in electrical wire circuits/electro-magnetic transmission lines, reverberations in the interior of the medium (i.e., wires or the like) cause ambiguity and loss of information bandwidth in the transmission of data. In a non-destructive evaluation and testing system, a setup is often modified from the desired design to avoid or reduce boundary reflections, which are a form of reverberations. Fundamentally, such reverberations are part-and-parcel of the wave-propagation governed by different physics but the same or similar wave-equations.

Propagating waves may be used in natural resource exploration, remote sensing, monitoring or surveillance, nondestructive testing, biological or medical diagnosis or treatment, communication, etc. In all these fields, eliminating/reducing reverberations may produce better data acquisition from the propagating waves.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In this application, methods and apparatuses that enable the construction of a special source/source wavefield (f1 solution) and utilize such special sources for use in many different technical fields are described.

In one embodiment, waves are generated by a source and propagate into a medium bounded by an upper space and a lower half space. In the embodiment, the source is at an upper level within the upper half space. At least one receiver is at the upper level, and it receives reflected responses by the medium. The f1 solution of the medium is obtained and the source is activated according to the f1 solution to generate waves which propagate into the medium and focus at a focal point without reverberation.

In some embodiments of the present invention, the f1 solution may be obtained by the system of sources and receivers in field or by computer simulation, if some reflection data at the upper level is available.

In one embodiment, the f1 solution of the medium is obtained by: (a) injecting a single downgoing pulse by the at least one source into the subsurface and recording the reflection responses by the at least one receiver; (b) time-reversing the recorded reflection response and re-injecting it back into a medium by the at least one source, and recording its reflection by the at least one receiver; (c) muting the recorded reflected time-reversed reflection response just before it re-focuses on the source pulse; and (d) time-reversing the muted reflected time-reversed reflection response and adding it just after the single downgoing pulse forming a new downgoing pulse. The method may include an optional iterative step, which repeats the above four steps.

Similar steps may be implemented in a computer simulation where the steps are performed by a computer,

The focal point can be anywhere in the bottom half space or within the medium if some travel time knowledge of the medium is known.

The special source with f1 solution convolved with its original source signature can be used to focus energy at a single focal point, causing material change to the medium at the focal point. The medium at the focal point may comprise biological material, in vivo biological structure, an earth formation, non-biological material or the like. The wave may be symbol carriers, seismic waves, ultrasonic waves, acoustic waves or the like.

In communication systems, once the f1 wavefield is constructed, it may be used for suppression of transmitted echoes or transmitted reverberations in waves transmitting data.

In remote sensing systems, the f1 wavefield may be used for suppression of transmitted waves or transmitted energy following an initial pulse or desired waveform (i.e., reverberations).

In seismic imaging or inversion applications, the f1 wavefield may be used as “virtual data.” The f1 wavefield can be used, optionally with the original data, in any of the following: in any variation of seismic migration imaging; in any waveform inversion; in reconstructing extended-image gathers of any kind by any existing imaging-condition methods; in applying any variation of migration velocity analysis or image-domain waveform inversion; in applying imaging or inversion methods for high-resolution targeted reservoir characterization by extracting a small subset of reconstructed “virtual data” enclosing a reservoir region of interest; or assuming the existence of two or more time-lapse surface data sets, applying any of the above-mentioned imaging and/or inversion methodologies for high-resolution targeted time-lapse reservoir monitoring, by extracting a time-lapse series of small subset of reconstructed “virtual data” enclosing a reservoir region of interest.

### BRIEF DESCRIPTION OF DRAWINGS

Embodiments of this disclosure are described with reference to the following figures. The same numbers are used throughout the figures to reference like features and components. A better understanding of the methods or apparatuses can be had when the following detailed description of the several embodiments is considered in conjunction with the following drawings, in which:

FIG. 1a illustrates a data acquisition system for a marine seismic survey;

FIG. 1b illustrates an ultrasound imaging data acquisition, processing and display system;

FIG. 1c illustrates a drilling system where a telemetric system is used;

FIGS. 2a, 2b and 2c illustrate the incomplete time-reversal paradigm in layered media;

FIGS. 3a-c illustrate transmitted and reflected waves in a single layer between two half spaces;

FIGS. 4A-F illustrate amplitudes in iterative time-reversal steps;

FIG. 5 illustrates a two-layer density and velocity model;

FIGS. 6A-J illustrate incomplete time-reversal iterations for f1 construction in the two-layer model;

FIG. 7 illustrates a 2D model with one non-uniform thickness layer between two half spaces;

FIG. 8 illustrate several results of the iterative time-reversal procedure;

FIG. 9 illustrates a comparison of the wavefield in the interior for the original focusing source including the transmitted multiples and the f1 source wavefield after 7 iterations from −0.20 seconds to 0.00 second;

FIG. 10 illustrate comparison of the wavefield in the interior for the original focusing source including the transmitted multiples and the f1 source wavefield after 7 iterations from −0.00 seconds to 0.15 seconds;

FIG. 11 illustrate comparison of the wavefield in the interior for the original focusing source including the transmitted multiples and the f1 source wavefield after 7 iterations from 0.20 seconds to 0.35 second;

FIG. 12 illustrates a flow chart for a method 1200 to construct f1;

FIG. 13 illustrates a modified flow chart for a method 1300 to construct f1;

FIGS. 14a-14e illustrate comparison of a communication system transmitting frequency coded symbols with and without f1 source; and

FIG. 15 illustrates a computer system which may implement parts of the methods described above.

### DETAILED DESCRIPTION

Reference will now be made in detail to embodiments, examples of which are illustrated in the accompanying drawings and figures. In the following detailed description, numerous specific details are set forth in order to provide a thorough understanding of the subject matter herein. However, it will be apparent to one of ordinary skill in the art that the subject matter may be practiced without these specific details. In other instances, well-known methods, procedures, components, and systems have not been described in detail so as not to unnecessarily obscure aspects of the embodiments.

It will also be understood that, although the terms first, second, etc., may be used herein to describe various elements, these elements should not be limited by these terms. These terms are only used to distinguish one element from another. For example, a first object or step could be termed a second object or step, and, similarly, a second object or step could be termed a first object or step. The first object or step, and the second object or step, are both objects or steps, respectively, but they are not to be considered the same object or step.

The terminology used in the description of the disclosure herein is for the purpose of describing particular embodiments only and is not intended to be limiting of the subject matter. As used in this description and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

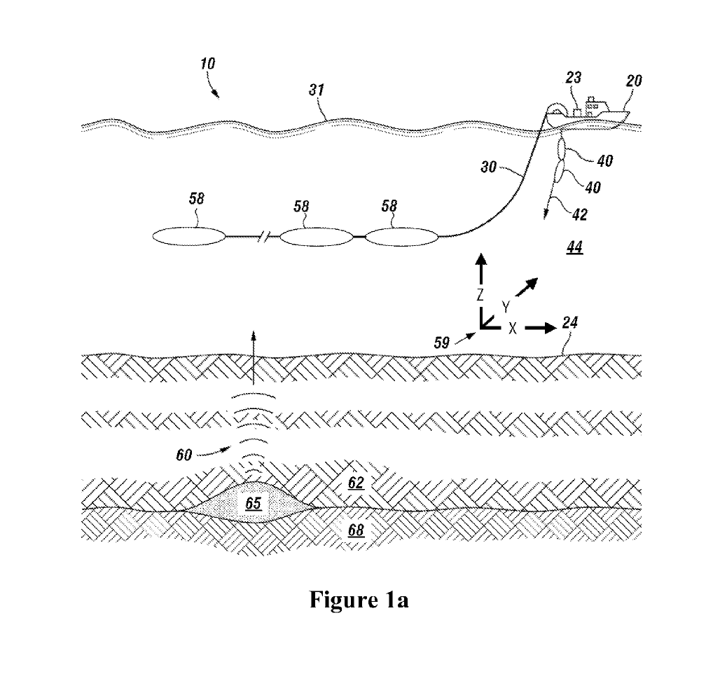

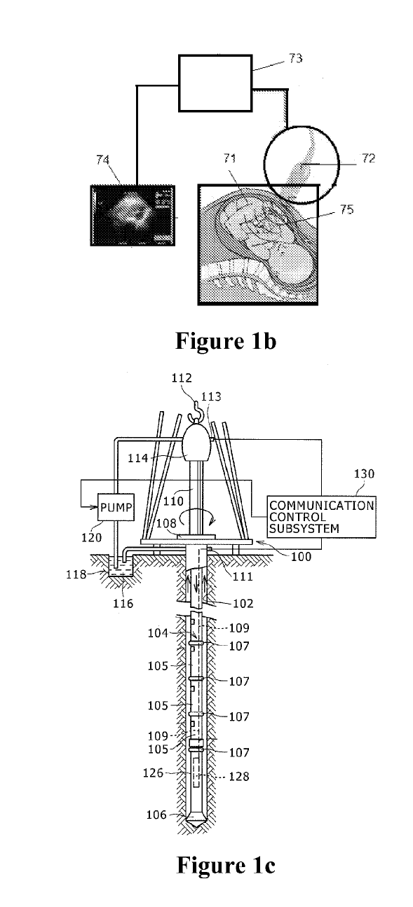

FIG. 1 illustrates several wave propagation systems in different industries. The data acquired are processed and used for various uses.

FIG. 1a illustrates a data acquisition system for a marine seismic survey. In the system 10, a survey vessel 20 tows one or more seismic streamers 30 (one streamer 30 being depicted in FIG. 1) behind the vessel 20. It is noted that the streamers 30 may be arranged in a spread in which multiple streamers 30 are towed in approximately the same plane at the same depth. As another non-limiting example, the streamers may be towed at multiple depths, such as in an over/under spread, for example.

The seismic streamers 30 may be several thousand meters long and may contain various support cables (not shown), as well as wiring and/or circuitry (not shown) that may be used to support communication along the streamers 30. In general, each streamer 30 includes a primary cable into which seismic sensors that record seismic data are mounted. The streamers 30 contain seismic sensors 58, which may be hydrophones to acquire pressure data, or multi-component sensors. For example, sensors 58 may be multi-component sensors; each sensor may be capable of detecting a pressure wavefield and at least one component of a particle motion that is associated with acoustic signals that are proximate to the sensor. Examples of particle motions include one or more components of a particle displacement, one or more components (inline (x), crossline (y) and vertical (z) components (see axes 59, for example)) of a particle velocity and one or more components of a particle acceleration.

The marine seismic data acquisition system 10 includes one or more seismic sources 40 (two seismic sources 40 being depicted in FIG. 1), such as air guns and the like. The seismic sources 40 may be coupled to, or towed by, the survey vessel 20. The seismic sources 40 may operate independently of the survey vessel 20, in that the sources 40 may be coupled to other vessels or buoys, as just a few examples.

As the seismic streamers 30 are towed behind the survey vessel 20, acoustic signals 42 (an acoustic signal 42 being depicted in FIG. 1), often referred to as “shots,” are produced by the seismic sources 40 and are directed down through a water column 44 into strata 62 and 68 beneath a water bottom surface 24. The acoustic signals 42 are reflected from the various subterranean geological formations (or targets), such as a formation 65 that is depicted in FIG. 1.

The incident acoustic signals 42 that are generated by the sources 40 produce corresponding reflected acoustic signals reflected by the targets, or pressure waves 60, which are sensed by the seismic sensors 58. It is noted that the pressure waves that are received and sensed by the seismic sensors 58 include “upgoing” pressure waves that propagate to the sensors 58 without reflection from the air-water boundary 31, as well as “downgoing” pressure waves that are produced by reflections of the pressure waves 60 from an air-water boundary 31.

The goal of the seismic acquisition is to build up an image of a survey area for purposes of identifying subterranean geological formations or targets, such as the geological formation 65. Subsequent analysis of the representation may reveal probable locations of hydrocarbon deposits in subterranean geological formations. Depending on the particular survey design, portions of the analysis of the representation may be performed on the seismic survey vessel 20, such as by the signal processing unit 23. In other surveys, the representation may be processed by a seismic data processing system.

FIG. 1b illustrates an ultrasound imaging data acquisition, processing and display system. The target 71 (a fetus) is to be imaged using a transducer 72, which includes both a source and a receiver. The transmitted signal and received reflection signal (ultrasound waves 75) from the transducer 72 are sent to a processor 73. The processor 73 collects and processes the signals and converts them into a human visible image 74 and displays the image 74 on a screen. A medical care-giver may use the image 74 to monitor the condition of the fetus. In this system, the primary wave is an ultrasound wave.

FIG. 1c illustrates a drilling system where communication between downhole equipment and surface controllers is performed by a telemetric system using wired drill strings/wired drillpipe.

As shown in FIG. 1c, a platform and derrick 100 are positioned over a borehole 102 that is formed in the earth by rotary drilling. A drill string 104 is suspended within the borehole 102 and includes a drill bit 106 at its lower end.

The drillstring 104 and drill bit 106 attached thereto are rotated by a rotating table 108 which engages a kelly 110 at the upper end of the drill string 104. The drillstring 104 is suspended from a hook 112 attached to a traveling block (not shown). The kelly 110 is connected to the hook 112 through a rotary swivel 114 which permits rotation of the drill string 104 relative to the hook 112. Alternatively, the drillstring 104 and the drill bit 106 may be rotated from the surface by a “top drive” type of drilling rig.

The drillstring 104 includes a bottom hole assembly (BHA) 126, which is mounted close to the bottom of the drillstring 104 proximate the drill bit 106. The BHA 126 generally includes capabilities for measuring, processing and storing information, and for communicating with the earth's surface, such as via a local communications subsystem 128 that communicates with a similar communications subsystem 130 at the earth's surface. One of the technologies that the local communications subsystem 128 uses to communicate with the surface communications system 130 is through the use of one or more communication channels provided by a wired drill pipe.

For instance, as shown in FIG. 1c, the drillstring 104 includes multiple sections of wired drillpipe 105 interconnected with couplers 107. Each section of wired drillpipe 105 contains one or more communication channels within the pipe, such as the communication channel 109 shown schematically in FIG. 1c. The couplers 107 are configured to mechanically couple the sections of wired drillpipe 105 to one another and to couple the sections of the communication channel(s) 109 so as to form a contiguous communication channel 109 from one end of the series of interconnected sections of wired drill pipe to the other end.

The lowermost end of the wired drillpipe 105 is coupled to a bottom hole assembly (BHA) 126 such that the local communications subsystem 128 can transmit and receive communications via the communication channel 109. The uppermost end of the wired drill pipe 105 is coupled through a coupler 111 to the surface communication subsystem 130. In this manner, the communication channel(s) 109 may be used to transmit signals (e.g., telemetry signals or data, command signals, etc.) between the surface and the BHA 128, as well as various other downhole components that may be coupled to the communication channel(s) 109.

The communication path between the downhole equipment and the surface has many intermediate sections, connectors or couplers where the impedances among the wires or the connectors are different. All the different wires or connectors may cause different reverberations in transmission or reflection. The reverberations may overlap with coded signals that may cause confusion, errors or loss of communication bandwidth.

For simplicity, all examples described below are related to seismic imaging in seismic exploration, in which the waves emitted by sources are reflected by the target and received by receivers. The subsurface media contains different layers and interfaces between the layers. The different layers cause undesirable internal reverberations that need to be dealt with or avoided. However, the methods are equally applicable to propagating wave systems that have interfaces in the wave traveling path in any arrangement, as long as the waves emitted by the sources are disturbed in some way by the target or interfaces and the disturbed waves are received by the receivers. The receivers can be on both sides. Some examples of non-seismic systems include at least, remote sensing with electromagnetic waves, biomedical imaging, non-destructive imaging and telecommunications.

The different waves (propagative or dissipative), sources, or receivers in different industries do not affect the wave propagation properties and the imaging processes. In seismic imaging, the wave is an elastic wave or an acoustic wave. The target is a subsurface geological structure. The sources are elastic or acoustic wave generators (e.g., airguns, vibrators) and the receivers are pressure or particle motion sensors (e.g., geophones, hydrophones, accelerometers or similar).

A datum as in “redatum” refers to a standard position or level that measurements are taken from. Data refers to the measurements or their representations in various formats. Data redatuming refers to a process in which the data are transformed as if the measurements are taken from a new location or a new level. Additionally, a “datum” here implies a surface that need not be horizontal and/or flat, and includes any geometrically conceivable surface.

### F1 Solution Construction

In seismic surveys, e.g., as shown in FIG. 1a, the source or receivers may only be accessed on one side, i.e., above the subsurface objects. There are so called data-driven focusing methods, which enable the creation of virtual source and receiver points in the subsurface using only the surface reflection response and an estimate of the background velocity model. As these methods do not require any actual sources or receivers in the subsurface, but still reconstruct the full waveform including all internal multiple scattering, these methods are often described as beyond interferometry. Furthermore, it has been shown that the sensitivity of these methods to the knowledge of the background velocity model is weaker than that of model-based approaches, suggesting that data created by these methods can be used consistently with the original reflection data in improved true-amplitude imaging and full-waveform inversion procedures.

Rose (Rose, J. H., 2002, ‘Single-sided’ Autofocusing of Sound in Layered Materials: Inverse Problems, 18, 1923) disclosed a proof of the iterative construction, for arbitrary inhomogeneous media, of the full waveform transmission Green's function in a one-dimensional (1D) system. Wapenaar et al. (e.g., Wapenaar, K., F. Broggini, E. Slob, and R. Snieder, 2013, Three-dimensional Single-Sided Marchenko Inverse Scattering, Data-Driven Focusing, Green's Function Retrieval, and their Mutual Relations: Phys. Rev. Lett., 110, no. 8, 084301; Wapenaar, K., E. Slob, J van der Neut, J. Thorbecke, R. Snieder, and F. Broggini, 2013, Three-Dimensional Marchenko Equation for Green's Function Retrieval “Beyond Seismic Interferometry”: SEG Tech. Prog. Exp. Abs., 4573-4578.) extended the solution to two-dimensional (2D) or three-dimensional (3D) systems and disclosed two so-called fundamental solutions, f1 and f2, which are special focusing wavefields.

The f1 solution as used in Wapenaar and the present disclosure refers to a focusing wave whose focal point is at the bottom interface, while the f2 solution is a focusing wave whose focal point is at the top interface. In this application, “focusing” refers to a scenario where the energy of a wave is concentrated in a single point in the interior for at least one moment in the propagation, and that point is not further illuminated by incident waves, i.e., the reverberations.

It has been shown by many authors that time-reversal is a natural, effective, and in many respects optimal way of constructing focusing wavefields in the interior of an inhomogeneous medium. Robust time-reversal of a multiply-scattered wavefield enables super-resolved focal spots compared to focusing in free-space. Furthermore, if measurements are available on a completely closed surface, time-reversal can undo the scattering at each interface (scatterer) by recombining reflected and transmitted (backward and forward scattered) wavefields, creating a single retro-focusing arrival with a focal spot only limited by the diffraction limit.

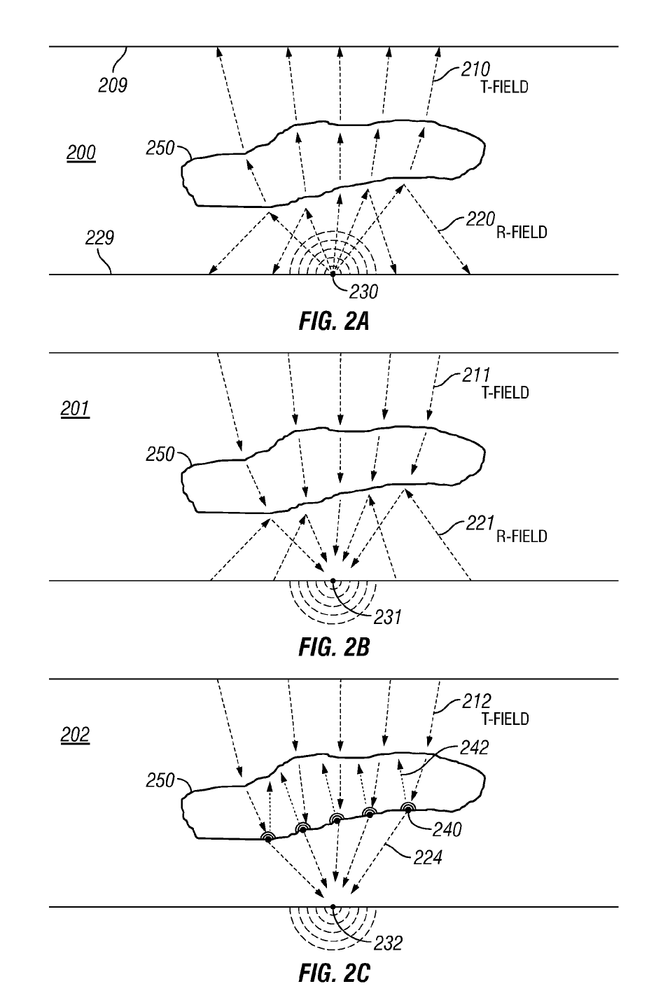

FIGS. 2a-2c illustrate some limitations of the time-reversal paradigm. FIG. 2a shows an upgoing impulsive wave source 230 below the inhomogeneous medium 250. The wavefield has transmitted and reflected waves 210 and 220. The transmitted wave 210 is sampled at an upper level (datum) 209 which is within a homogenous upper half space, and the reflected wave 220 is sampled at bottom level (datum) 229 which is within a homogeneous bottom half space. The locations of 209 and 229 can be any convenient location. FIG. 2b (Middle) shows that if both the T-field 210 and the R-field 220 are recorded and time-reversed (the reversed fields 211 and 221), a purely downgoing impulsive wave 231 below the inhomogeneous medium 250 is produced. In FIG. 2c (Bottom), if only the T-field 210 is recorded and time-reversed (212), the time-reversal is incomplete and secondary sources 240, arising from the missing time-reversal of the R-field 220, produce waves 224 that eventually reflect downward and invalidate the construction of f1. The missing R-field 220 is the equivalent of the addition of such secondary sources 240 wherever the back propagating wavefield is not complete.

In the time-reversal paradigm, it is straightforward to see how a directional source wavefield below an arbitrary inhomogeneous overburden can be created. For example, as illustrated in FIGS. 2a and 2b (top and middle panel), a purely downgoing pulse 230 below a stack of layers 250 can be created by time-reversing the reflected and transmitted fields due to a particular purely upgoing wave, namely that purely upgoing wave which, when time-reversed, would produce the desired downgoing wave after propagating through the focus. However, the limitations of the time-reversal paradigm become apparent when considering the problem of f1 construction.

Per definition, f1 is the wavefield that produces a single downgoing pulse below an arbitrary inhomogeneous stack of layers/overburden, constructed using only up- and downgoing waves at the top of the stack/above the overburden. Therefore, this wavefield cannot be the result of such a time-reversal, as the waves reflected downward from the inhomogeneous stack/overburden for such a source are not available in such a setting (see FIG. 2c, bottom panel; R-field 220 is not available). In other words, the f1 wavefield is not the time-reverse of a wavefield due to a purely upgoing impulsive source below the inhomogeneous stack of layers as the downward reflections of such a wavefield are by definition absent from f1.

It is recognized that f1 is specifically constructed such that all downgoing waves apart from the direct downgoing pulse are eliminated from the response. In this sense, f1 is not a natural wavefield as it suppresses the natural forward (in this case downward) scattering that occurs in any layered/inhomogeneous medium. As noted above, there are additional waves 242 that can be thought of as generated by secondary sources 240 when the whole field is not available in the time-reversal, as shown in FIG. 2c, denoted by dashed lines 242 and small semi-circles 240, respectively. In embodiments of the present disclosure, such incomplete time-reversal and the secondary sources arising from it, may be used in the construction of f1.

Due to the special nature of f1, it is useful to consider first how f1 might be constructed with explicit knowledge of the medium. For simplicity, the following description is based on a 1D medium, but the insights/results/deductions may be applied in accordance with embodiments of the present disclosure to arbitrary inhomogeneous media.

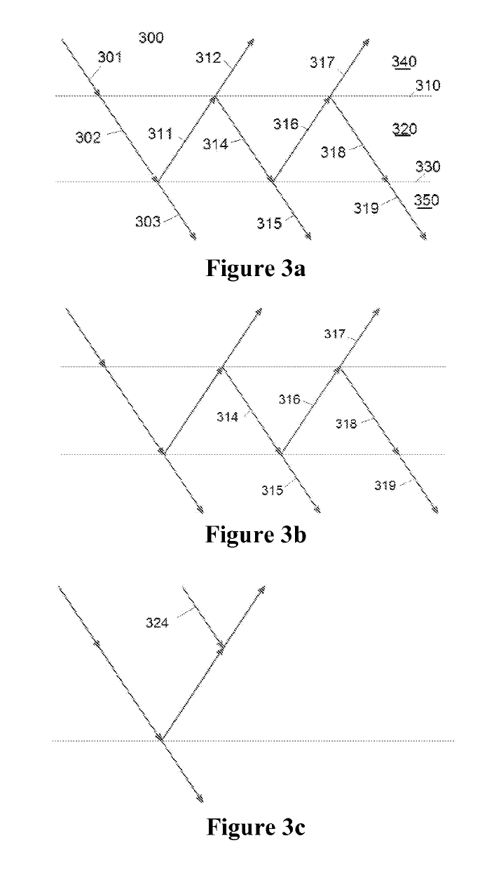

First, the simplest case 300 for f1 construction, namely single layer 320 embedded between two half spaces (340 and 350), as shown in FIG. 3, is considered. The upper level and the bottom level are selected to be at the upper interface 310 and the bottom interface 330. As discussed in reference to FIGS. 2a, this selection of levels is for simplicity and convenience and not for necessity, i.e., it is not a limitation. The two levels can be located anywhere in the two half spaces 340 and 350, respectively.

For a single downgoing incident pulse 301 from above, the transmitted response contains an infinite number of reverberations. It is clear that all the downward reflections at the top interface must be eliminated in order to construct f1. However, it is also interesting to note that all unwanted downward reflections (314-319) ultimately can be traced back to the initial single downward reflection 314 from the underside of the top interface 310. As the amplitude of the preceding upgoing wave and the reflection coefficient (from below) at the first interface are fixed, the only means to suppress the unwanted downward reflection is to send a well-timed, appropriately-scaled additional downgoing incident pulse 324 from above, which arrives exactly at the same time at the first interface 310 (from above) as the upgoing event 314 that creates the initial downward reflection. This may be done, in accordance with embodiments of the present disclosure using explicit knowledge of the medium and, as shown below, also without any explicit knowledge of the medium except for the reflection response. Thus, in one embodiment of the present invention, using only a single additional well-timed and well-scaled incident pulse from above, emitted sometime between the initial pulse and the corresponding upgoing arrival, with transmitted amplitude equal, but opposite to the reflected amplitude of the initial downward reflection, an infinite number of reverberations and all downgoing waves below the second interface can be eliminated apart from the initial downgoing pulse, as shown in FIG. 3c.

It may seem counter-intuitive that an infinite number of downgoing events below the second interface are eliminated using only a finite number (in some embodiments only a single) of additional well-timed and well-scaled downgoing arrivals. In other words, in aspects of the present disclosure, for a single-layer system, there may be only two (2) events (one original event and one additional event). It can be shown that the number of events m(N) needed for an N-layer system is:

m(N)=1+Σₙ₌₁^{N}(m(N−n−1)),

where m(0)=1, m(−1)=1. For example, for a 3-layer system, we need 8 events, i.e., we need to add 8−1=7 additional events to remove all (and infinite number of downgoing) events below the last interface.

It is noted from FIGS. 3a-c, in one embodiment of the present disclosure, that the timings of the downgoing events designed to cancel the unwanted downgoing reflections are the times needed to reach the downward reflection point, where the unwanted waves are generated, minus the times needed to reach the same downward reflection point from the surface. In a 1D medium, this time is straightforward to compute: for example in the case of the single layer between two half-spaces, denoting the first time as a+b+b, where a is the one-way time from the surface down to the reflection point, and b as the one-way time from the reflection point down to the deepest interface, it is clear that the desired timing is simply 2b. This may be estimated in a general 1D medium if layer thicknesses and average interval velocities are available.

However, note that to time-reverse the reflected wavefield (excluding the direct downgoing source pulse and the transmitted field), this wavefield would reflect off the layers in the subsurface exactly at the time when the unwanted (and wanted) reflections occurred and arrive back at the surface exactly at the time of the required additional events (and similarly for the unwanted event's multiples).

In embodiments of the present disclosure, the reason that time-reversal of the reflected wavefield provides events with exactly those timings of the required additional events is that the time-reversal described above is incomplete. It is so because it excludes time-reversal of the missing transmitted wavefield. If the missing transmitted wavefield had been included in the time-reversal then the time-reversal would have been complete and those reflected waves with the correct kinematics of the required additional events would not have been generated because they are not present in the original reflected and transmitted wavefield. In embodiments of the present invention, this principle is referred to as incomplete time-reversal and it is exploited/used to generate events with initially incorrect amplitudes, but correct timings, to attenuate the so-called unwanted downward reflections.

It is noted that in embodiments of the present disclosure, the absence of time-reversed transmitted waves, which originally constituted the unwanted downward reflections, provide through the process of incomplete time-reversal, exactly waves arriving with the timings needed to attenuate such transmitted waves.

More specifically, in accordance with an embodiment of the present disclosure, the following iterative method 1200 can be used for f1 construction in a 1D medium:

Step 1210, Inject a single downgoing pulse into the subsurface and record the reflection response r(t) (this is a forward iteration step);

Step 1220, Time-reverse the recorded reflection response, re-inject it back into the medium, and record its reflection (this is an incomplete time-reversal using only reflection response, no transmission response; this is a reverse iteration step and this is different from the forward iteration step 1210);

Step 1230, Mute the recorded reflected time-reversed reflection response just before it re-focuses on the initial source pulse;

Step 1240, Time-reverse the muted reflected time-reversed reflection response and add it just after the original single downgoing pulse; and

Step 1250, Repeat steps 1210-1240 until convergence, each time replacing the previous downgoing pulse with the new downgoing pulse computed in step 1240.

The embodiments described above comprising incomplete time-reversal also hold for arbitrary inhomogeneous 2D and 3D media, provided that most of the energy indeed is radiated away from the inhomogeneous zone upward or downward and not sideways. The same method may be used with only a minor modification in steps 1 and 3 for a 2D or 3D media, as discussed below.

It is noted that after step 1240, a new downgoing pulse is obtained. If this new downgoing pulse is injected into the medium, the reverberation due to the medium will be reduced, depending on the transmission/reflection coefficients of the medium. In many cases, the reduction in reverberation may be sufficient such that the iterative step 1250 is optional and not needed. In many cases, an alternative predefined stopping criterion, e.g., a finite number of iteration, may be used instead of convergence. It can be shown that the iterative procedure outlined above also constructs/converges to right amplitudes and this provides further insight into the physics of incomplete time-reversal.

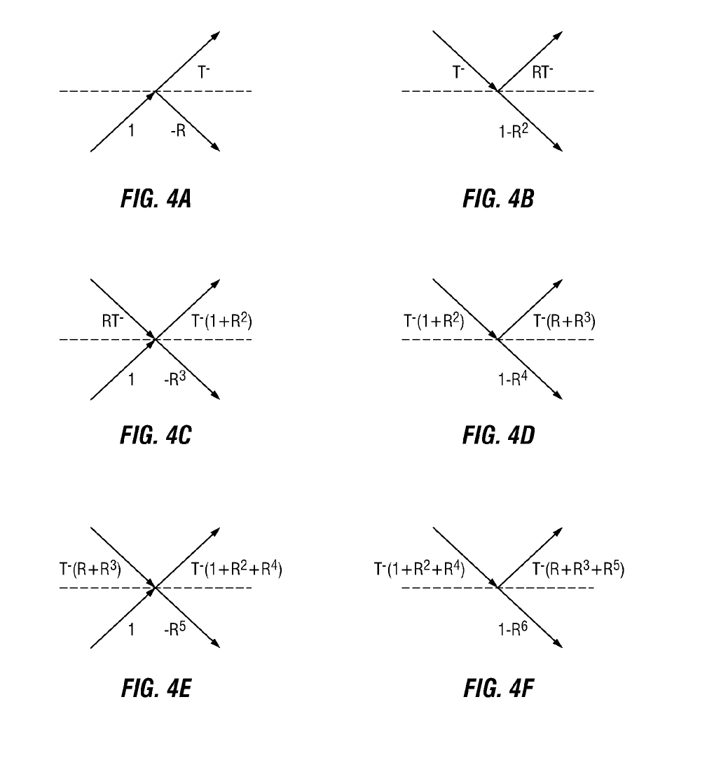

Referring to FIGS. 4a-4f, the case of a single interface is considered. In the figures, the reflection coefficient from above is defined as R, the transmission coefficient from above is defined as T⁺ and the transmission coefficient from below is defined as T⁻. In the illustrated example, the reflection coefficient from below is −R and: T⁺T⁻=1−R². Without loss of generality, the incident wave from below is considered to have an amplitude equal to 1.

Thus, the initial recorded data has an event with amplitude T⁻ and the unwanted reflection has amplitude −R. Next the data is iterated using the incomplete time-reversal algorithm given above. Note that according to step 1240 of the algorithm given above, on the forward steps, the new source pulse has some iteration quantity (to be clarified below) plus the original source pulse. Thus, on forward runs, marked f in the following table, the incident wavefields will contain the original amplitude 1 contribution due to the original source pulse. Also, note that because the analysis is for a single interface, there is no need for a muting step as described above. With these preliminaries, a few iterations may be considered. Each iteration is also illustrated visually as a subplot in FIGS. 4a-4f. Incomplete time-reversal iterations are marked r:

| | | | | Scattered | |
|---|---|---|---|---|---|
| Iter | Inc. below | Incident above | Scattered up | down | FIG. |
| 1f | 1 | 0 | T⁻ | −R | 4a |
| 1r | 0 | T⁻ | T⁻ R | 1 − R² | 4b |
| 2f | 1 | T⁻ R | T⁻ (1 + R²) | −R³ | 4c |
| 2r | 0 | T⁻ (1 + R²) | T⁻ (R + R³) | 1 − R⁴ | 4d |
| 3f | 1 | T⁻ (R + R³) | T⁻ (1 + R² + R⁴) | −R⁵ | 4e |
| 3r | 0 | T⁻ (1 + R² + R⁴) | T⁻ (R + R³ + R⁵) | 1 − R⁶ | 4f |

It is noted that T⁺ does not appear anywhere in the table because all the wavefields that are transmitted downward are proportional to T⁻and hence their downward transmission results in factors of 1-R² as per the identity given above. From the outputs of these iterations, the following observations may be made:
With each pair of reverse and forward runs, the transmitted wavefield reduces in amplitude by a factor R². After two pairs of reverse and forward runs (i.e., when iter.=3f) the downward reflected wave has been reduced in amplitude from its initial value of −R, via −R³, to −R⁵. Clearly, this will converge quadratically to the desired value of 0 for the downward reflected wave.
With each pair of forward and reverse runs, the time-reversed wavefield transmitted back into the subsurface approaches the desired value of 1 quadratically, with the error term reducing by a factor of R² per pair of forward and reverse runs also. After two pairs of forward and reverse runs (i.e., when iter.=3 r) the error in the back propagating wavefield amplitude below the interface has reduced from −R², via −R⁴, to −R⁶.
The amplitude of the additional event required to cancel the downward reflection is proportional to the amplitude of the upgoing wave that created the unwanted downward reflection, modified by the upward transmission coefficient of the interface and sum of odd powers of the reflection coefficient.

The above observations explain intuitively why the correct amplitudes are recovered with the iterative incomplete time-reversal scheme of the present disclosure as described above. Note that the second observation also explains why this method works in a more complicated (1D) medium having many layers and interfaces as it is observed that with each pair of forward and reverse runs, the amplitude of the waves time-reversed through the first interface approach the amplitude (1 in this case) of the upgoing wave causing the unwanted downward reflection. This suggests that the algorithm strips off the unwanted downward reflections in a top-down iterative manner.

As with the kinematic description given above, the reasoning given above also applies to the case of more complicated 2D and 3D inhomogeneous media, with the same provisions as before that the wavefield radiated from the inhomogeneous medium should be propagating predominantly in the up- and downgoing directions and not sideways.

Before proceeding with some examples, two slight variations on the 1D algorithm given above are reviewed. Firstly, it is shown how in one aspect the algorithm should be updated to produce a particular arrival at a particular focusing time, given an estimate of the background velocity model. This also explains how in an aspect of the present disclosure, a single downgoing (energy focused) pulse can be obtained at an arbitrary point inside the medium, in-line with the focusing definitions given in the introduction, which generalizes the algorithm, at least for 1D, to the case of (energy) focused arrivals in the interior. Secondly, it is described how, in an aspect of the present disclosure, the algorithm(s) may be updated in the case of 2D and 3D processes.

Both the kinematic and dynamic arguments presented above did not make reference to any knowledge of the model, including any background velocity model. This was done to emphasize that this knowledge is not needed, per se, for the construction of wavefields focusing like f1. However, strictly speaking, the wavefield defined as f1 should also focus at t=0 s. Also, to generalize the method to construction of (energy) focused pulses inside the inhomogeneous medium or 2D and 3D, it may be necessary to proceed with making such knowledge about the background velocity model explicit in the algorithm.

### Focusing Inside the Medium and at Time Zero

Defining the background velocity model as c₀=c₀(x), the focusing time t_{f} corresponding to a focus point x_{f} is given by:

$${t}_{f} = {\int }_{0}^{{x}_{f}} \frac{1}{{c}_{0} ( x )} d x .$$

Thus, with this definition, to produce a focus at t=0, (with x_{f} possibly inside the medium), the algorithm in accordance with an aspect of the present disclosure may be modified as follows (modifications shown in bold):

Step 1310, Inject a single downgoing pulse into the subsurface at t=−t_{f} and record the reflection response r(t);

Step 1315, Mute the reflection response for t>t_{f}, when primary reflections from all the interfaces above the desired focus have arrived;

Step 1320, Time-reverse the recorded muted reflection response, re-inject it back into the medium, and record its reflection (incomplete time-reversal);

Step 1330, Mute the recorded reflected time-reversed muted reflection response just before it re-focuses on the initial source pulse at t=t_{f};

Step 1340, Time-reverse the muted reflected time-reversed reflection response and add it just after the original single downgoing pulse at t=−t_{f};

Step 1350, Repeat steps 1310-1340 until convergence, each time replacing the previous downgoing pulse with the new downgoing pulse computed in step 1340.

This produces a (n energy) focus at location x_{f} in the medium and time t=0. Talking through the changes, we can see that in step 1310, the initial downgoing source pulse is now injected at t=−tⱼ. Since the (energy) focus results from the original downgoing transmitted source pulse, it must be that this pulse is emitted at t=−t_{f} in order to produce the focus at t=0. Next, a new step 1315 is added to the algorithm, which mutes the reflection response for times t>t_{f}. As t_{f} corresponds to the one-way time down to the focus point, and the original wavefield was emitted at t=−t_{f} and focuses at t=0, muting after t=t_{f} corresponds to muting any events that arrive after the primary reflection from a depth maximum to the focus depth. As is clear from the description in the section on direct construction of f1, muting after t=t_{f} provides that all events generating downward reflections above the focus point have arrived at (transmitted to) the surface. Recall that these transmissions are needed in the incomplete time-reversal step to generate the additional events needed to start canceling the downward reflection on the next forward run. In step 1320, the time-reversed muted reflection response is injected into the medium and its reflection recorded. The result of the time-reversal step is then itself muted (in step 1330) at a time just before re-focusing on the initial source pulse. This step, hence, essentially remains the same, except that now a time for the refocusing at the surface can be given, which is at t=t_{f}. The resulting muted trace is then time-reversed and added back to the original source pulse as previously, at time t=−t_{f}, and constitutes the source pulse for the next iteration.

The updated iteration described above, in accordance with an embodiment of the present invention, is valid for creating energy focused pulses below the inhomogeneous stack of layers (by choosing t_{f} larger than the transmission time through the full stack of layers) as well as inside the stack of layers (by choosing t_{f} between 0 and the full direct transmission time).

The above iterative construction of f1 and amplitudes are equally valid for 2D and 3D media with a few minor changes. Similar to the discussion related to method 1200, the iteration step 1350 is also optional.

First, to produce an initial focused pulse, including subsequent reverberations, below or in an inhomogeneous 2D or 3D medium, in an embodiment of the present disclosure, more than one surface source is necessary. Thus, an array of surface sources, appropriately sampled to avoid aliasing, and long enough to asymptotically include all sideways scattering arrivals, is needed. Second, besides the depth of the focus, the horizontal location impacts the wavefield needed to produce the initial focus. Thus it is convenient to define the location of the focus through the arrival times of the direct wave from the focus point to each of the sources at the surface. Thus, in the description of the algorithm, terms like t=−t_{f} need to be replaced by terms like t(x|x_{f})=−t_{f}(x|x_{f}), and where x denotes a (range of) point(s) at the surface and x_{f} denotes the desired location of the focus. Second, to perform incomplete time-reversal, the wavefield is recorded everywhere along the surface, spanning at least the same range as the sources. Thus, the array of receivers spans or coincides with the array of sources (like transducers in an ultrasonic acoustic experiment). Third, the mute in step 1315 of the algorithm remains symmetric in time around t=0. Fourth, the mute in step 1330 also uses the spatially variant initial source pulse time. These changes, as provided in some embodiments of the present disclosure, produce the desired results in 2D and 3D.

### Numerical Examples

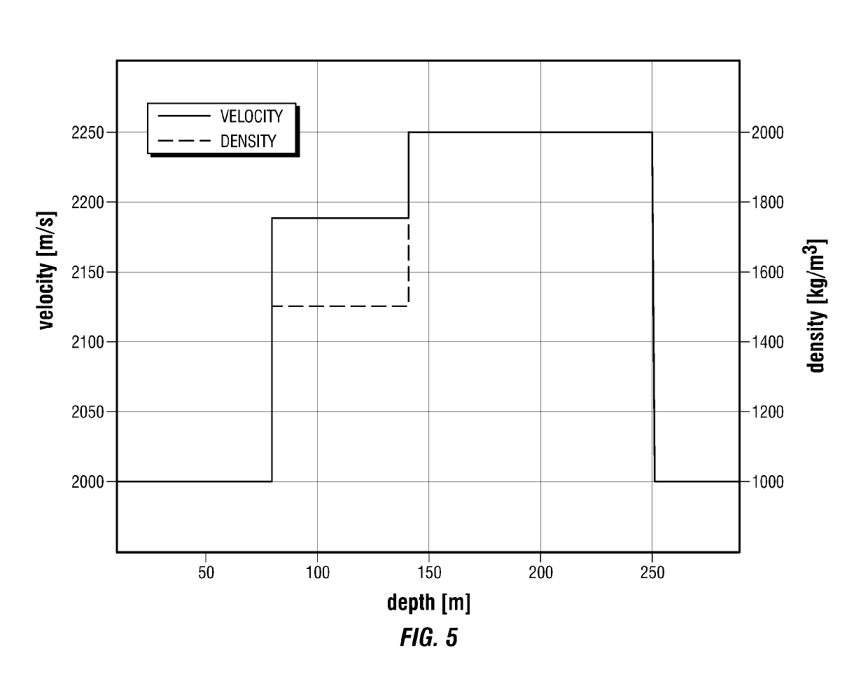

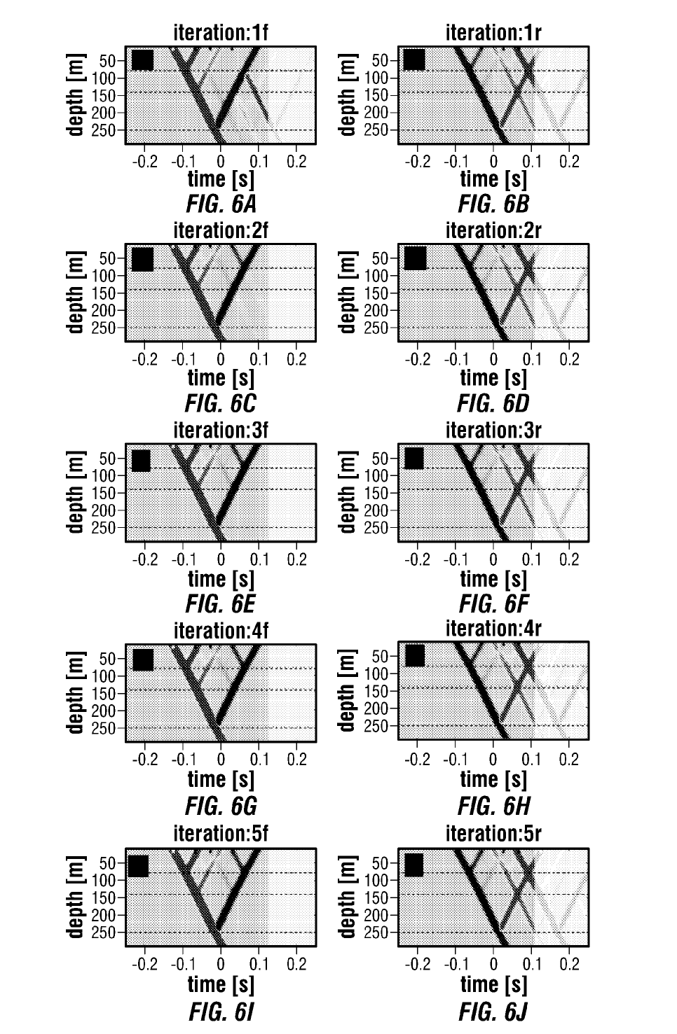

FIG. 5 illustrates a 1D 2-layer model and FIGS. 6a-6j illustrate the corresponding incomplete time-reversal iterations for f1 construction.

The system shown in FIG. 5 has two layers between two half spaces. The wave propagation in this model for a source pulse having a single downgoing event is shown in FIG. 6a. The wave propagation is displayed in a VSP-style plot, also called a waterfall plot, which is convenient for displaying the wavefield for all depths (vertical) and all times (horizontal) simultaneously. Note the infinite number of transmitted arrivals following the directly transmitted source pulse due to the reverberations of the waves in the two layers (e.g., at a depth of 275 m). The reflected wavefield at 15 meters depth, which is also the source depth, is then used to start the iterative time-reversal scheme as explained previously. The chosen one-way transmission time to create a focus at t=Os was t_{f}=0.125 s, which, according to the velocity model shown, should produce a focus without reverberations below the deepest interface. The source pulse, at t=−t_{f}, is marked with a white square, as is the expected refocusing time of the source-pulse on the time-reversal runs at t=t_{f}. The resulting wave propagation for each forward and reverse iteration is shown in FIGS. 6b to 6j.

Thus, the reflected wavefield at 15 meters depth in FIG. 6a is time-reversed muted (as indicated by the semi-transparent regions) and used as the source wavefield in iteration 1 r. Note that the time-reversed wavefield is initially traveling back into the subsurface with the original amplitudes and signs (i.e., events dipping to the right in the top right panel at 50 meter depth). However, when the time-reversed wavefield reaches the first interface, because the downward transmitted part is excluded from the time-reversal, the wavefield does not retrace its original path exactly, unscattering at each interface. Instead, the time-reversal symmetry is broken (If the time-reversal was perfect/complete, then it would be expected that the left and right pictures to be mirror images of each other, mirrored exactly in the t=t_{f} vertical axis.) and the waves reflect from the interfaces at every location where the time-reversal is incomplete (see also FIG. 2c). These reflected waves are recorded at the surface and occur exactly at the times needed, in a forward simulation, to start canceling the unwanted downward reflections from the underside of all but the last interface, as explained in the section on iterative time-reversal. The expected times of the additional events (computed directly from the model) are indicated with black squares (three, in this case, as expected).

Still looking at FIG. 6b, even though the time-reversal is incomplete, it still produces somewhat of a focus at t=t_{f} because, although the amplitudes are not correctly reconstructed, kinematically, all waves still arrive at the same time at the original source location. Furthermore, the waves continue to arrive after t=t_{f}, due to the fact that the time-reversal is incomplete and the fact that there is no inverse source acting to absorb the energy from the focusing arrivals. In some aspects, it may be important for the iterative time-reversal procedure to mute the wavefield just before t=t_{f} on the time-reversal runs since any events following t=t_{f} otherwise would appear before the original downgoing source pulse (at t=−t_{f}) in the next forward iteration and generate new downward reflections, which again would have to be cancelled, etc. Also, the wavefield may be muted just before t=t_{f} as the original source pulse is included according to step 1340 of the iterative time-reversal.

In the next forward iteration, (FIG. 6c), the downward reflections are already significantly attenuated. From the right panels it may be seen that only a finite number of additional events are needed to eliminate the downward reflections on the forward runs as the infinite reverberations at earlier times in the time-reversed wavefield quickly disappear. By iterating this process, an increasingly improved estimate of f1 is obtained (left panels) until in the bottom left panel, no other transmitted arrivals can be discerned other than the initial downgoing direct source pulse.

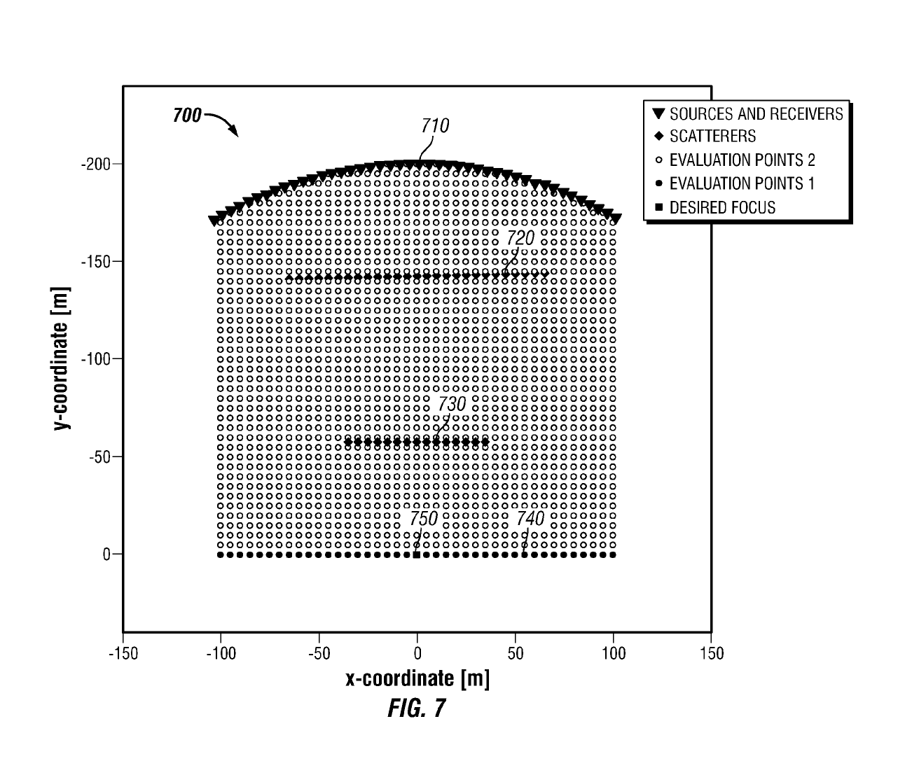

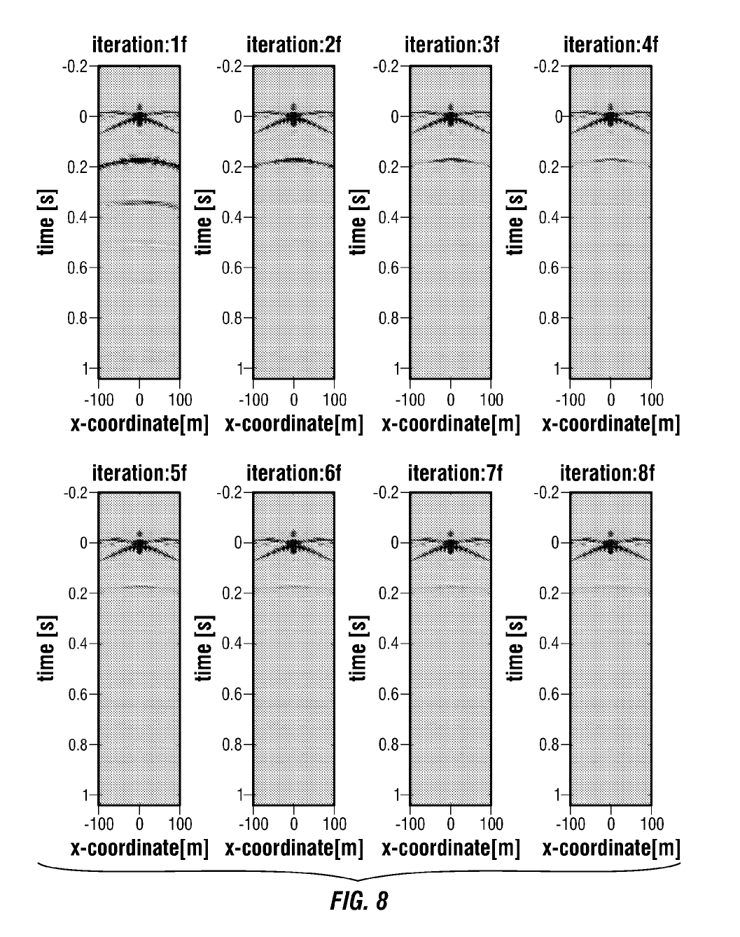

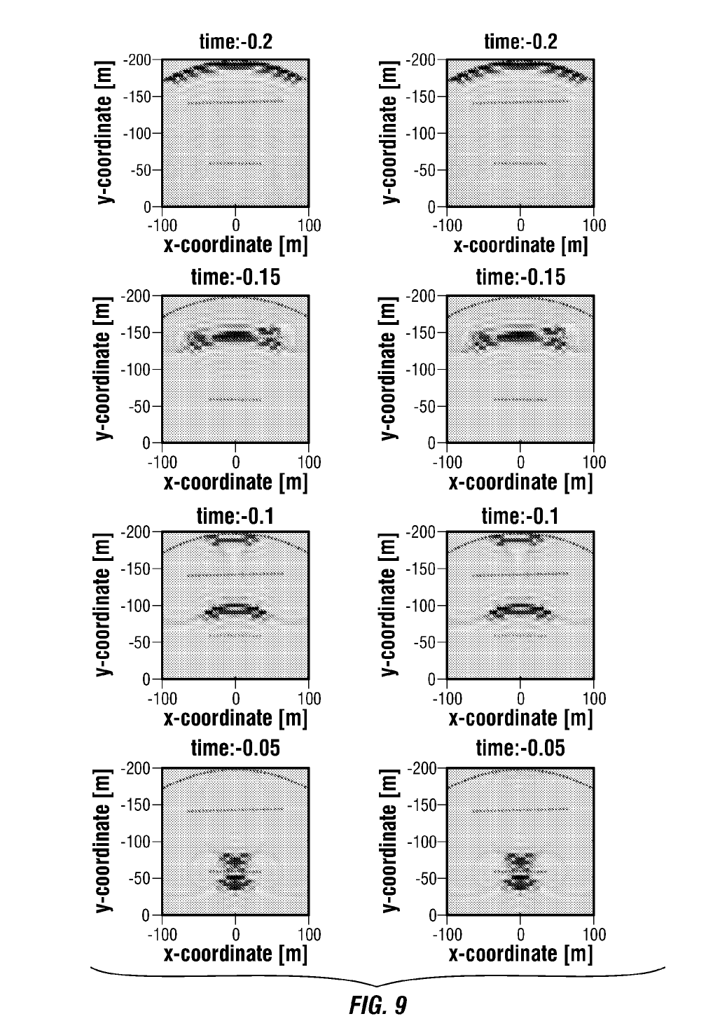

FIG. 7 illustrates a 2D one-layer example which has non-uniform thickness between two half spaces. FIG. 8 illustrates results of the iterative time-reversal procedure on the model shown in FIG. 7 after every pair of iterations, evaluated at a line of horizontal receivers through the desired focus point. Note the dramatic reduction of transmitted multiples after 7 iterations. FIGS. 9-11 illustrate comparison of the wavefield in the interior for the original focusing source, including the transmitted multiples (left column) and the f1 source wavefield after 7 iterations (right) for various time between −0.2 seconds to +0.2 seconds.

It is noted that the model uses closely spaced point scatterers to simulate two non-parallel interfaces. This is because, in some embodiments, the Foldy modeling method is used to compute the data, which is limited to configurations of point scatterers. Also the sources and receivers were chosen to lie on a segment of a circle around the desired focus point for the f1 construction. This was done to facilitate the muting in the iterative time-reversal scheme: since the data recorded from a time-reversal run need to be muted just before the moment the waves refocus onto the initial source wavefield, which occurs at a constant time since the sources & receivers are equidistant from the desired focus point, reducing the muting operation to muting everything using the same time across the array. This is merely used in an example of one embodiment of the present disclosure and is not a requirement for the methods described herein.

To evaluate the result of the iterative time-reversal, a row of receivers 740 going through the desired focus point 750 was defined. The result of the iterative time-reversal procedure, after evaluating the transmitted wavefield at the red receiver 740 locations after every pair of reverse and forward iterations, is shown in FIG. 8. In the example of the present disclosure, most of the transmitted reverberations are effectively attenuated using only seven reverse and forward iterations, leaving only the desired focal spot associated with transmission of the direct, focusing wavefield.

To further qualitatively evaluate the results of the iterative time-reversal, the f1 wavefield (after seven iterations) was computed/processed in the interior of the medium; namely, on the dense grid of receivers 740 in FIG. 7. Twelve snapshots of this wavefield are shown in the right columns of FIGS. 9-11. These f1 wavefield snapshots are shown alongside (i.e., in the left columns) the original focusing wavefield as it propagates and reverberates in the interior. In the first four snapshots (i.e., FIG. 9), not many differences can be seen between the f1 wavefield and the original focusing wavefield. This is because the downward reflection from the underside of the top row of scatterers still has to happen, and, in fact, the upgoing reflected wavefield that would be the cause of that downward reflection is still far enough away from the top row of scatterers that no additional cancellation wavefield has yet been generated.

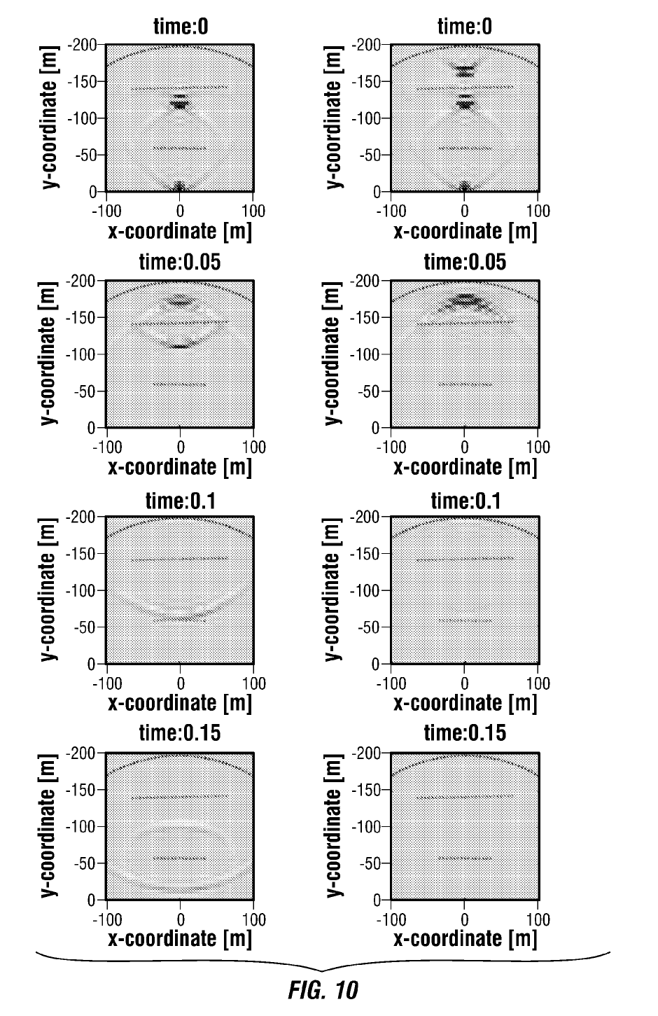

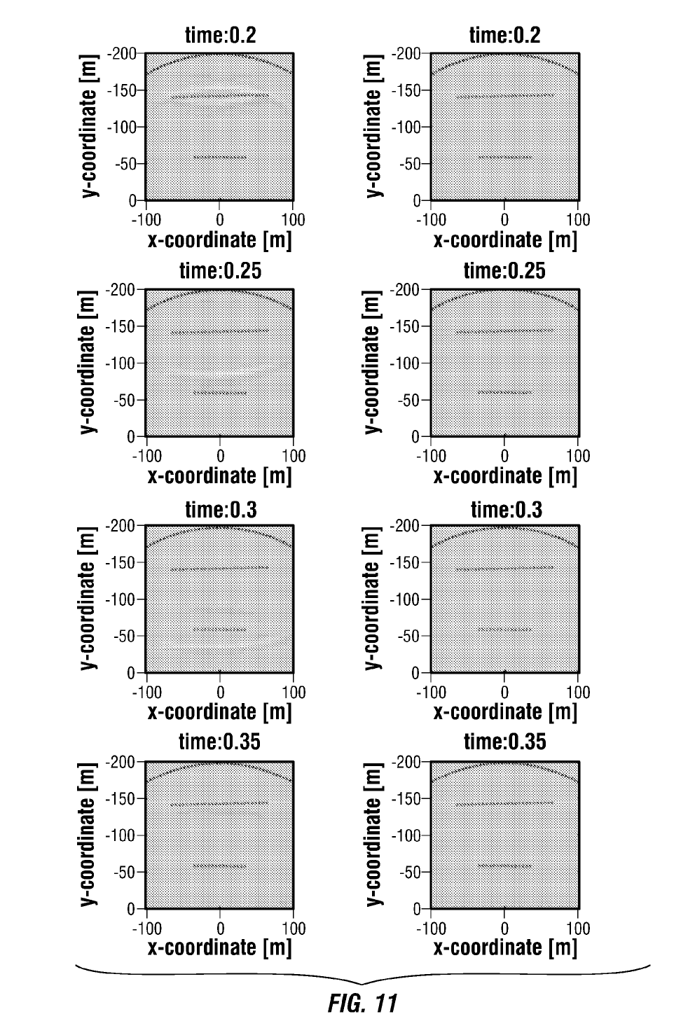

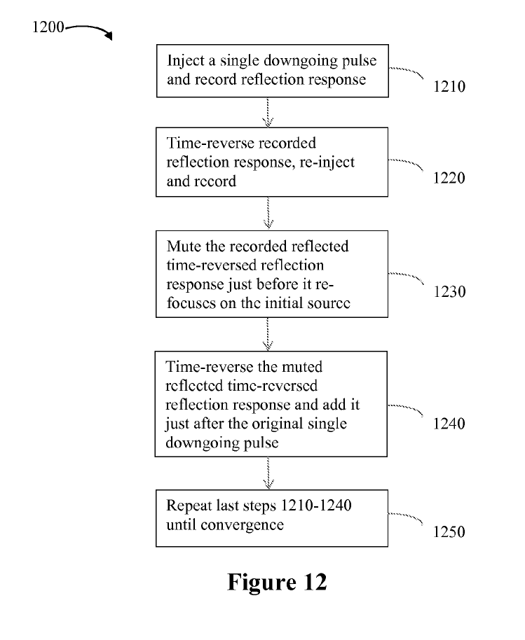

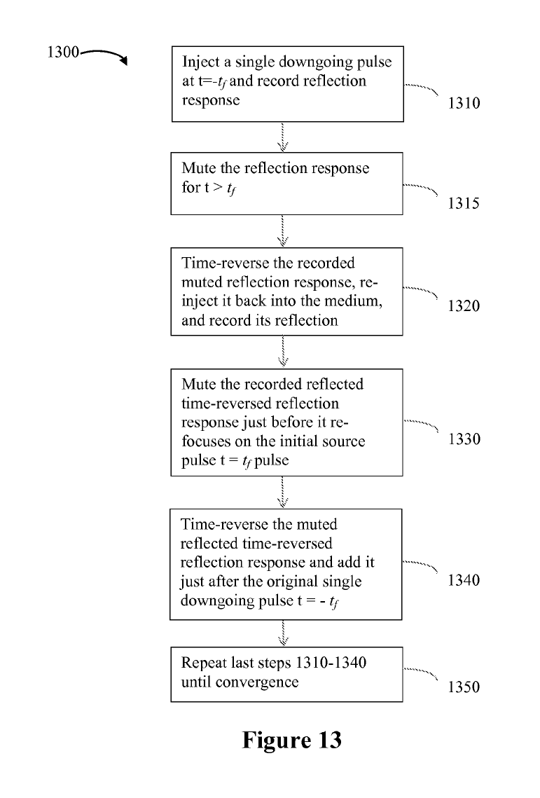

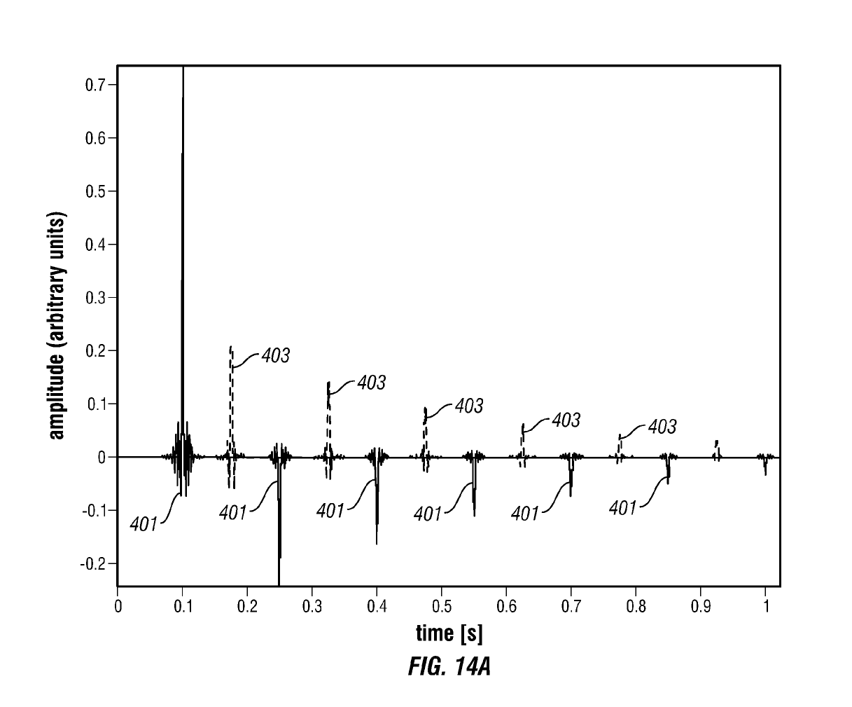

In FIG. 10, the first snapshot shows the wavefield focusing at the desired location (left and right); it also shows the cancelling wavefield that has been generated as part of f1, which has started to propagate downward toward the top row of scatterers, where it should be transmitted and will cancel the impending downward reflection from the top row of scatterers. This can be seen in the 2nd snapshot (for t=0.05 s) in FIG. 10, where, on the left, the original downgoing reflection can be seen, but on the right, the downgoing reflection has been cancelled by the additional events in the f1 wavefield. Snapshots 5 and 6 confirm that the unwanted downward reflection has been cancelled as it shows no sign of the downward reflection in the f1 wavefield in the right column, even though the downward reflection can be seen clearly interacting and being transmitted through to the desired focus location in the left column. Even in the last 4 snapshots (9 to 12) in FIG. 11, the reverberation of the downward reflection can be seen clearly in the left column, including its transmission through to the focus, whereas no such waves can be seen in the f1 wavefield in the right column. Thus, it may be concluded from this simple 2D example that the iterative time-reversal method according to one embodiment of the present application works as expected.

As shown in the examples, the iterative incomplete time-reversal methods in accordance with embodiments of the present invention can reconstruct an f1 solution, which can focus energy to a point without unwanted reverberations.

### f1 Applications

The f1 fundamental solution of the wave-equation producing a single transmitted pulse below an arbitrary reverberative stack of inhomogeneous layers was scrutinized Both model-driven as well as data-driven constructions were discussed and the concept of incomplete time-reversal was introduced to facilitate the iterative data-driven construction. Thus, in accordance with one embodiment, the f1 wavefield may be generated without any knowledge of the subsurface other than the reflection response.

It was also shown how the amplitudes converge to the correct values using the iterative TR scheme. In one aspect, a modification of the algorithm, requiring additional knowledge of a smooth background velocity model, may guarantee focusing at time-zero (strictly reproducing f1) and also enable construction of focused energy pulses in the interior of the medium. Both a 1D and a 2D example were given. The iteration can be done offline, or online (i.e., by iterating the acquisition with a waveform that updates between iterations). The incomplete time-reversal concept provides an intuitive explanation how such a particular transmission wavefield can be generated simply from the reflection response, and the concept may also provide insight into this aspect for the data-driven transmission Green's function.

When data from sources and receivers are available at a level (e.g., 209 or 310), then, in accordance with embodiments of the present disclosure, using the above discussed methods, f1 solution can be constructed or simulated with a computer.

With the construction of f1 functions, in embodiments of the present disclosure, a true-amplitude full-wave response (i.e., the complete Green's function) between sources may be retrieved at the real acquisition surface and “virtual” receivers/sensors inside the subsurface at any desired location from the original data with both real sources and receivers at the acquisition surface. By reciprocity, the true-amplitude full-wave response (i.e., the complete Green's function) between receivers at the real acquisition surface and “virtual” sources inside the subsurface at any desired location can be obtained from the original data with both real sources and receivers at the acquisition surface. This allows for redatuming an actual recorded response to a desired depth in the subsurface. These responses may constitute “virtual data” for sources and receivers at any locations in the subsurface.

Based on the retrieved fields or “virtual data,” many imaging and inversion applications can be processed in accordance with embodiments of the present disclosure. These may include:
1. Given the reconstructed “virtual data,” and optionally the original data in conjunction, applying any variation of seismic migration imaging (e.g., reverse-time, beam or Kirchhoff migration) and accounting for both transmission and reflection data from enclosing boundaries. In such methods, the enclosing boundaries (i.e., top and bottom) may come from either the original data boundary plus a boundary of “virtual data;” or two boundaries of “virtual data” enclosing a desired target area;
2. Given the reconstructed “virtual data,” and optionally the original data in conjunction, applying any variation of waveform inversion (e.g., full-waveform inversion, finite-frequency traveltime inversion, WKBJ-based AVO inversion) and accounting for both transmission and reflection data from enclosing boundaries. In such methods, the enclosing boundaries (i.e., top and bottom) may come from either the original data boundary plus a boundary of “virtual data;” or two boundaries of “virtual data” enclosing a desired target area;
3. Based on items 1. and 2., and given the reconstructed “virtual data” and optionally the original data in conjunction, reconstructing extended-image gathers of any kind (e.g., angle-domain common-image gathers, subsurface offset and time-lag gathers, extended model gathers) by any existing imaging-condition methods (e.g., adjoint-state methods, source-receiver interferometric imaging, multidimensional deconvolution), accounting for both transmission and reflection data from enclosing boundaries. The enclosing boundaries (i.e., top and bottom) may come from either the original data boundary plus a boundary of “virtual data;” or two boundaries of “virtual data” enclosing a desired target area;
4. Based on item 3., and given the reconstructed “virtual data,” and optionally the original data in conjunction, applying any variation of migration velocity analysis or image-domain waveform inversion (e.g., angle-domain residual-moveout tomography, differential semblance-based, finite-frequency annihilator-based), accounting for both transmission and reflection data from enclosing boundaries. The enclosing boundaries (i.e., top and bottom) may come from either the original data boundary plus a boundary of “virtual data;” or two boundaries of “virtual data” enclosing a desired target area;
5. Based on items 1. through 4., applying any of the above-mentioned imaging and/or inversion methodologies for high-resolution targeted reservoir characterization by extracting a small subset of reconstructed “virtual data” enclosing a reservoir region of interest; and
6. Based on items 1. through 4., and assuming the existence of two or more time-lapse surface data sets, applying any of the above-mentioned imaging and/or inversion methodologies for high-resolution targeted time-lapse reservoir monitoring by extracting a time-lapse series of small subset of reconstructed “virtual data” enclosing a reservoir region of interest.

f1 solution may also be constructed using the above methods in the field with physical sources and receivers. As illustrated in FIG. 7, to deliver energy that is focused at focal point 750, sources and receivers can be arranged as in 710. Following one of the methods discussed above, in an embodiment of the present disclosure, one source may be activated to generate a downgoing pulse signal into the medium, and the receivers may record the reflection signals. The reflection responses may be time-reversed and re-injected via the sources back into the medium and recorded. The recorded reflected time-reversed reflection responses may be muted just before they re-focus on the initial source pulse. Then, lastly, the muted reflected time-reversed reflection responses may be time-reversed and added just after the original single downgoing pulse to form a new pulse. The new pulse obtained in this way, in accordance with an embodiment of the present invention, is an approximate or imperfect f1 solution. If the method is repeated until convergence is obtained, then the last pulse is the perfected f1 solution.

In an embodiment of the present invention, once an f1 solution is obtained, whether an approximate solution or a perfect solution, the sources are activated according to this pulse (which is essentially the convolution of the original source signature and the f1 solution), and a wave field according to the f1 solution is created in the medium where the energy is focused at focal point (e.g., 750) without reverberation.

The methods presented above can construct f1. These methods are quite general and may have applications in many different fields, even if governed by different physics. For example, in electrical engineering and communications there is the concept of an electrical transmission line, which is a specially designed cable for transmitting high frequency (radio) waves without being affected by reflections from discontinuities in the cable such as connectors and joints. Great effort is made to match the impedance along the entire length of such cables. Using an embodiment of the present disclosure, the presented construction can make it possible to transmit radio frequency waves along the cable without being affected by transmitted multiples. Similar transmission line concepts also occur in downhole telemetry applications and in long distance telephony. Therefore the f1 construction methods may be used.

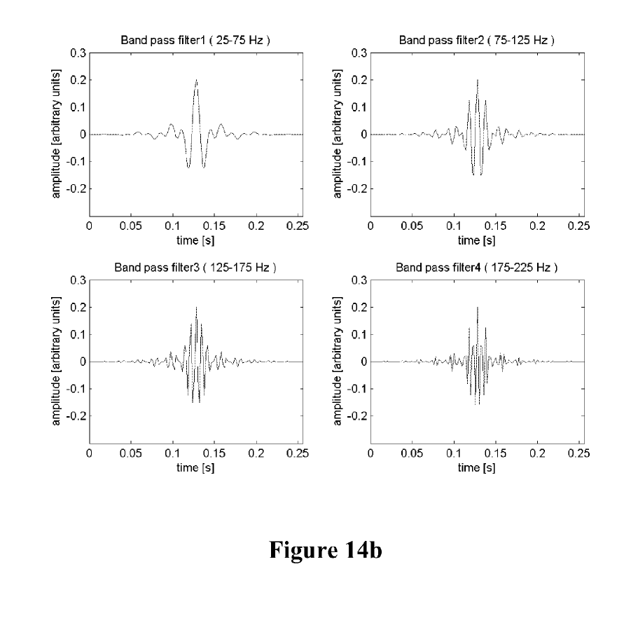

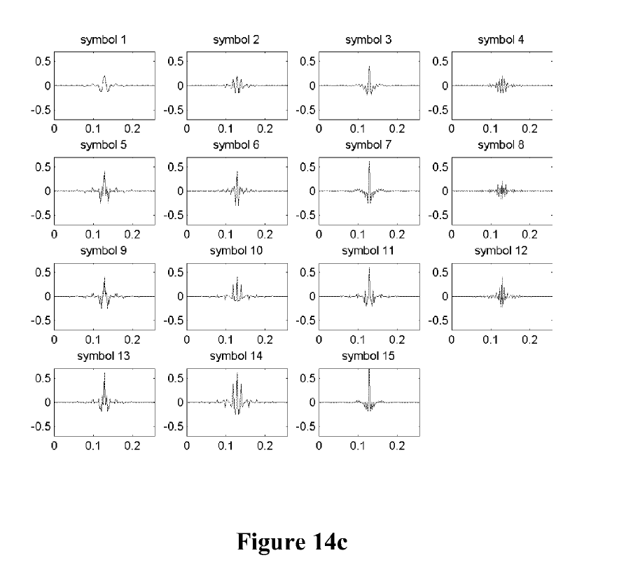

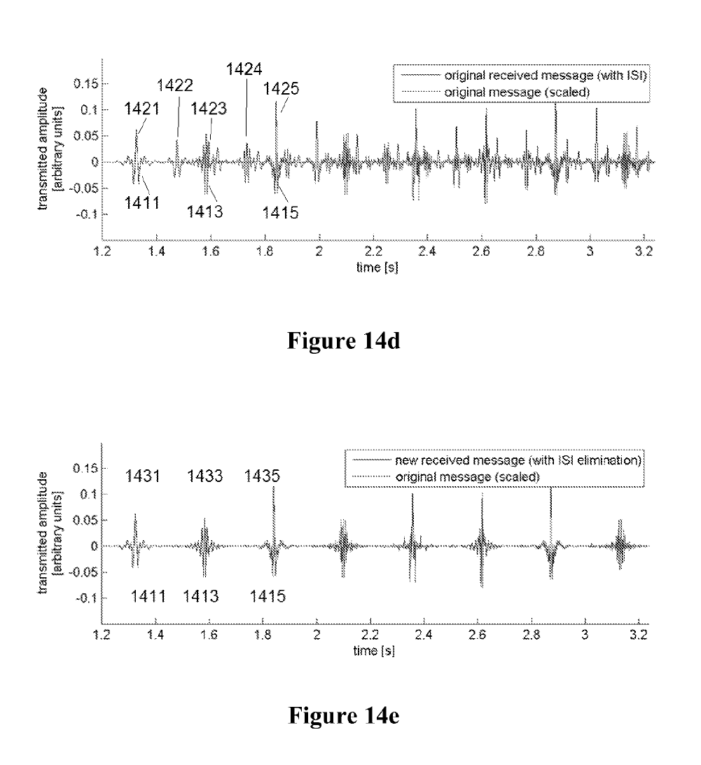

FIGS. 14a-14d illustrate comparison between a communication system transmitting frequency coded symbols with or without reverberation, which may cause inter-symbol interference (ISI). In this example, the medium is a simple three-layer medium:

| | Layer 1 | Layer 2 | Layer 3 |
|---|---|---|---|
| Velocity (m/s) | 2000 | 2000 | 2000 |
| Density (kg/m3) | 2000 | 200 | 2000 |
| Thickness (m) | 100 | 150 | 100 |

The layers 1 and 3 are two half spaces. The thicknesses of these two layers refer to the distances of sources and receivers to the reverberative layer 2. FIG. 4a shows the reflection Green's function 1401 and the transmission Green's function 1403 of this medium. It can be seen from FIG. 14a that the transmitted signal has strong multiples (or reverberations) every 0.15 seconds. If the symbol duration is of the same order as the reverberation time then significant inter-symbol interference will be generated.

For an illustrative purpose, four band pass filters are used as shown in FIG. 14b. Each has the same order of duration as the reverberation time in the transmission Green's function as in FIG. 14a. Fifteen symbols can be coded using the four band pass filters excluding the zero symbol, as shown in FIG. 14c. The 15 symbols are created by summing different combinations of these bandpass filters (e.g., symbol 3 is formed by adding filters 1 and 2 and symbol 11 by adding filters 1, 2, and 4). Note that the time-length of these symbols is also of the same order as the reverberation time in the transmission Green's function. Therefore, there will be significant ISI transmitting a string of these symbols (i.e., a message) through the reverberative medium.

FIG. 14d shows an original message (symbols 1411, 1413 and 1415) overlapped with the received message (symbols 1421, 1422, 1423, 1424 and 1425) through the medium. It is clear that there are numerous extra messages (symbols 1422 and 1424) due to the reverberation. Without additional information or processing, the received message will not be decodable.

FIG. 14e shows the same original message (symbols 1411, 1413 and 1415). Instead of transmitting the message as is, it is convolved with an f1 solution of the medium, which is constructed with one of the above discussed methods. Once the original message is convolved, the reverberation due to the reverberative medium is eliminated. The new received message (symbols 1431, 1433 and 1435) with ISI elimination is almost the same as the original message (symbols 1411, 1413 and 1415). By convolving the original message before transmission, the original message is transmitted and received intact.

It is noted that the iterative construction does not increase the focusing power or the power of the initial downgoing pulse, but instead removes subsequent transmitted multiples which create ambiguity and complexity. As illustrated in the example shown in FIGS. 7-11, energy can be delivered to one focal point without secondary focal points or other reverberation with only the rough knowledge of the medium (e.g., the background velocity). However, with the ability to focus energy to a single focal point without unwanted secondary focal points, the energy delivered can be increased substantially. The focused high energy source is advantageous in many situations, e.g., non-invasive surgical applications. This focused high energy source can deliver high energy to a single focal point and cause material change to anything at the focal point. In a surgical application, the medium at the focal point is biological tissues and the material change can be evaporation, fusion or otherwise destruction of the tissues.

This feature of no secondary events may also be desirable for some seismic/oilfield applications, e.g., for targeted stimulation. Research into such targeted stimulation for EOR using synchronized sweeping vibrators was done in the 1980's, although it appears to have been largely unsuccessful. Using the f1 construction methods, the targeted stimulation with sweeping vibrators is feasible. In a targeted stimulation, the medium at the focal point is an earth formation and the material change can be the compression, squeezing or dilatation, breakdown or fracture of the formation at the focal point

In the exploration seismic domain, a common occurrence is an overburden or shallow near-surface that scatters the downgoing source wavefield significantly and effectively complicates the effective source wavefield with which the subsurface is being probed. Provided the reflection response from such a shallow subsurface can be reasonably demarcated, the above presented construction may also be used to construct an incidence wavefield that is significantly cleaner and should result in a less ambiguous way of probing the subsurface and subsequently provide lower complexity responses/seismic signals/data. Similarly, the f1 construction may also be useful in probing through casing in downhole formation evaluation.

Yet another seismic application of the methods disclosed herein is to use the f1 construction to transmit energy through layered basalts. Although the power of the transmitted source pulse is not improved, the efficiency as expressed in terms of the signal-to-transmitted multiples ratio is improved and effective source pulse complexity is reduced.

As discussed above, the f1 solution can be obtained via computer simulation when some reflection data at a datum are available. If not, f1 solution can be obtained directly from properly arranged sources and receivers, like the arrangement shown in FIG. 7.

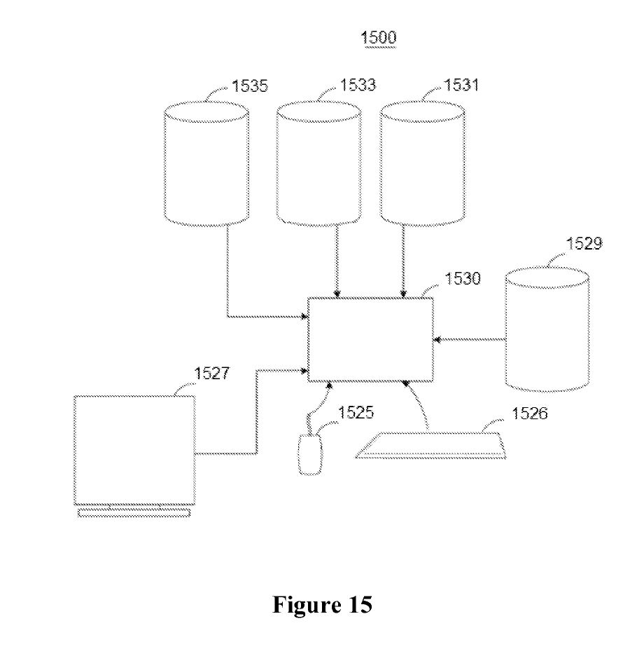

As those with skill in the art will understand, one or more of the steps of methods discussed above may be combined and/or the order of some operations may be changed. Further, some operations in methods may be combined with aspects of other example embodiments disclosed herein, and/or the order of some operations may be changed. The process of measurement, its interpretation, and actions taken by operators may be done in an iterative fashion; this concept is applicable to the methods discussed herein. Finally, portions of methods may be performed by any suitable techniques, including on an automated or semi-automated basis on computing system 1500 in FIG. 15.

The methods described above are typically implemented in a computer system 1500, one of which is shown in FIG. 15. The system computer 1530 may be in communication with disk storage devices 1529, 1531, 1533 and 1535, which may be external hard disk storage devices. It is contemplated that disk storage devices 1529, 1531, 1533 and 1535 are conventional hard disk drives, and as such, will be implemented by way of a local area network or by remote access. Of course, while disk storage devices are illustrated as separate devices, a single disk storage device may be used to store any and all of the program instructions, measurement data, and results as desired.

In one implementation, data from the receivers may be stored in disk storage device 1531. Various data from different sources may be stored in disk storage device 1533. The system computer 1530 may retrieve the appropriate data from the disk storage devices 1531 or 1533 to process data according to program instructions that correspond to implementations of various techniques described herein. The program instructions may be written in a computer programming language, such as C++, Java and the like. The program instructions may be stored in a computer-readable medium, such as program disk storage device 1535. Such computer-readable media may include computer storage media. Computer storage media may include volatile and non-volatile, and removable and non-removable media implemented in any method or technology for storage of information, such as computer-readable instructions, data structures, program modules or other data. Computer storage media may further include RAM, ROM, erasable programmable read-only memory (EPROM), electrically erasable programmable read-only memory (EEPROM), flash memory or other solid state memory technology, CD-ROM, digital versatile disks (DVD), or other optical storage, magnetic cassettes, magnetic tape, magnetic disk storage or other magnetic storage devices, or any other medium which can be used to store the desired information and which can be accessed by the system computer 1530. Combinations of any of the above may also be included within the scope of computer readable media.

In one implementation, the system computer 1530 may present output primarily onto graphics display 1527, or alternatively via printer 1528 (not shown). The system computer 1530 may store the results of the methods described above on disk storage 1529, for later use and further analysis. The keyboard 1526 and the pointing device (e.g., a mouse, trackball, or the like) 1525 may be provided with the system computer 1530 to enable interactive operation.

The system computer 1530 may be located at a data center remote from an exploration field. The system computer 1530 may be in communication with equipment on site to receive data of various measurements. The system computer 1530 may also be located on site in a field to provide faster feedback and guidance for the field operation. Such data, after conventional formatting and other initial processing, may be stored by the system computer 1530 as digital data in the disk storage 1531 or 1533 for subsequent retrieval and processing in the manner described above. While FIG. 15 illustrates the disk storage, e.g., 1531 as directly connected to the system computer 1530, it is also contemplated that the disk storage device may be accessible through a local area network or by remote access. Furthermore, while disk storage devices 1529, 1531 are illustrated as separate devices for storing input data and analysis results, the disk storage devices 1529, 1531 may be implemented within a single disk drive (either together with or separately from program disk storage device 1533), or in any other conventional manner as will be fully understood by one of skill in the art having reference to this specification.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope of the invention. Accordingly, the protection sought herein is as set forth in the claims below.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. §112, paragraph 6 for any limitations of any of the claims herein, except for those in which the claim expressly uses the words ‘means for’ together with an associated function.

1. A method for generating waves propagating into a medium bounded by an upper level and a bottom level, wherein at least one source is at or above an upper level and at least one receiver is at the upper level, wherein reflected responses by the medium are received by the at least one receiver, the method, comprising:
(a) obtaining an f1 solution for the medium; and
(b) activating the at least one source according to the f1 solution to generate waves wherein the waves propagate into the medium, wherein the waves focus at a focal point without reverberations.

2. The method of claim 1, wherein obtaining an f1 solution of the medium comprises:
(a) injecting a single downgoing pulse by the at least one source into subsurface and recording the reflection responses by the at least one receiver;
(b) time-reversing the recorded reflection response and re-injecting it back into the medium by the at least one source, and recording its reflection by the at least one receiver;
(c) muting the recorded reflected time-reversed reflection response just before it re-focuses on the source pulse; and
(d) time-reversing the muted reflected time-reversed reflection response and adding it just after the single downgoing pulse forming a new downgoing pulse.

3. The method of claim 2, wherein obtaining an f1 solution of the medium further comprises:
(e) repeating steps (a)-(d) until convergence or a predefined stopping criterion is reached, wherein in repeating the steps in step (a) of the repeated step a previous downgoing pulse is replaced with a new downgoing pulse as obtained in step (d).

4. The method of claim 1, wherein obtaining an f1 solution of the medium comprises:
performing by a computer an iterative incomplete time-reverse f1 solution construction comprising:
(a) injecting a single downgoing pulse into subsurface and recording the reflection responses;
(b) time-reversing the recorded reflection response and re-injecting it back into the medium, and recording its reflection;
(c) muting the recorded reflected time-reversed reflection response just before it re-focuses on the source pulse; and
(d) time-reversing the muted reflected time-reversed reflection response and adding it just after the single downgoing pulse forming a new downgoing pulse.

5. The method of claim 4, wherein obtaining an f1 solution of the medium further comprises:
(c) repeating steps (a)-(d) until convergence or a predefined stopping criterion is attained, wherein in each iteration the previous downgoing pulse is replaced with the new downgoing pulse as obtained in step (d).

6. The method as in claim 1, wherein the waves focused at the single focal point cause material changes to the medium at the focal point.

7. The method as in claim 6, wherein the medium at the focal point is biological tissues or earth formations.

8. The method as in claim 1, wherein the waves generated by the at least one source are symbol carriers.

9. The method as in claim 1, wherein the waves generated by the at least one source are seismic waves; and wherein the waves propagating within the medium have substantially no internal multiple reflections.

10. A computer implemented method for processing measurements of reflected responses of waves propagating into a medium bounded by an upper level and a bottom level, wherein at least one source is at or above a upper level and at least one receiver is at the upper level, wherein reflected responses by the medium are received by the at least one receiver, the method, performed by a computer, comprising:
(a) obtaining an f1 solution of the medium; and
(b) activating the at least one source according to the f1 solution to generate waves wherein the waves propagate into the medium, wherein the waves focus at a focal point without reverberations.

11. The method as in claim 10, wherein obtaining an f1 solution of the medium comprises:
(a) injecting a single downgoing pulse into subsurface and recording the reflection responses;
(b) time-reversing the recorded reflection response and re-injecting it back into the medium, and recording its reflection;
(c) muting the recorded reflected time-reversed reflection response just before it re-focuses on the source pulse; and
(d) time-reversing the muted reflected time-reversed reflection response and adding it just after the single downgoing pulse forming a new downgoing pulse.

12. The method of claim 11, wherein obtaining an f1 solution of the medium further comprises:
(e) repeating steps (a)-(d) until convergence or a predefined stopping criterion, each time replacing the previous downgoing pulse with the new downgoing pulse obtained in step (d).

13. The method as in claim 11, wherein a subsurface object has a focal time t_{f}, which is the time for a wave traveling from the upper level to a focal depth x_{f},
wherein (a) injecting a single downgoing pulse into the subsurface is injected at time t=−t_{f};
wherein (c) muting the recorded reflected time-reversed reflection response just before it re-focuses on the source pulse is muted at time t=−t_{f};
wherein (d) time-reversing the muted reflected time-reversed reflection response and adding it just after the single downgoing pulse is at time t=−t_{f}; and
further comprising muting the reflection response for t>t_{f}, when primary reflections from all the interfaces above a desired focus have arrived.

14. The method as in claim 13, wherein the focal depth is within or below the object.

15. The method as in claim 13, wherein the object is in a 1D, 2D or 3D system.

16. The method as in claim 13,
wherein the object is a geophysical target (65), a biological target (71), a remote-sensing target or a non-invasive investigation target.

17. The method as in claim 10, further comprising:
from the wavefields, retrieving true amplitude full-wave responses by receivers at desired locations within the medium or below the medium, wherein the retrieved true amplitude full wave responses are virtual data.

18. The method as in claim 17,
wherein the desired locations are on an enclosing boundary of an object,
the method further comprising:
applying a seismic migration imaging process on the virtual data accounting for both transmission and reflection data; or
applying a waveform inversion process on the virtual data accounting for both transmission and reflection data.

19. The method as in claim 18, the method further comprising:
reconstructing extended-image gathers with the virtual data accounting for both transmission and reflection data; or
applying a migration velocity analysis or image domain waveform inversion with the virtual data accounting for both transmission and reflection data.

20. The method as in claim 19, the method further comprising:
selecting a subset of the object and the corresponding virtual data; and
applying an imaging or inversion process for high-resolution targeted characterization on the selected subset.

21. The method as in claim 20, wherein second measurements of reflected responses recorded by receivers at a second time, the method further comprising:
processing the second measurements with the method of claim 1 to obtain a second virtual data at the second time;
selecting a subset of the object and the corresponding second virtual data;
applying an imaging or inversion process for high-resolution targeted characterization on the selected subset for the second time; and
comparing the two high-resolution targets for time-lapse monitoring.

22. A system for delivering waves propagating into a medium bounded by an upper level and a bottom level without reverberation within the medium, the system comprising:
at least one source is at or above the upper level;
at least one receiver at the upper level; and
a controller controlling the operation of the source,
wherein the controller has an f1 solution of the medium; and
wherein the controller controls the activating the at least one source according to the f1 solution to generate waves wherein the waves propagate into the medium, wherein the waves focus at a focal point without reverberations.

23. A data processing system for processing measurements of reflected responses of waves propagating into a medium bounded by an upper level and a bottom level, the system comprising:
at least one processor and at least one computer readable storage wherein the computer readable storage comprises computer executable instructions, which when executed by the processor, causes the processor to:
(a) obtain an f1 solution of the medium; and
(b) activate the at least one source according to the f1 solution to generate waves wherein the waves propagate into the medium, wherein the waves focus at a focal point without reverberations.
